(19) United States
(12) Patent Application Publication
(10) Pub. No.: US 2004/0147828 A1
(43) Pub. Date: Jul. 29, 2004

(54) Telescoping tip electrode catheter

(76) Inventor: Charles A. Gibson, Malden, MA (US)

Correspondence Address: DARBY & DARBY P.C., P. O. BOX 5257, NEW YORK, NY, 10150-5257, US

(21) Appl. No.: 10/475,618

(22) PCT Filed: Apr. 22, 2002
(86) PCT No.: PCT/US02/12663

(51) Int. Cl. A61B 5/04; A61B 18/14

(52) U.S. Cl. 600/374; 606/41; 607/122

(57) ABSTRACT

A medical device including a catheter and a telescoping mandrel with a tip electrode extending therethrough, the telescoping mandrel being moveable relative to the catheter shaft to extend from the catheter shaft distal end, thereby positioning the tip electrode at a distance from the catheter shaft distal end.

### FIELD OF THE INVENTION

This invention relates to medical devices for performing diagnostic, mapping, ablation, and other procedures and, more particularly, to a medical device including a telescoping tip electrode.

### BACKGROUND OF THE INVENTION

Catheters are often used in medical procedures to provide physical access to remote locations within a patient via relatively small passageways, reducing the need for traditional invasive surgery. The catheter tube also can be inserted into an artery or other passageway through a relatively small incision in the patient's body, and threaded through the patient's system of blood vessels to reach the desired target.

Various types of catheters are used in various procedures, both diagnostic and therapeutic. One general type of catheter used for both diagnostic and therapeutic applications is a cardiac electrode catheter. The diagnostic uses for a cardiac electrode catheter include recording and mapping of the electrical signals generated in the course of normal (or abnormal) heart function. Therapeutic applications include pacing, or generating and placing the appropriate electrical signals in order to stimulate the patient's heart to beat in a specified manner, and ablation. In an ablation procedure, electrical or radio-frequency energy is applied through an electrode catheter to form lesions in a desired portion of the patient's heart, for example the right atrium. When properly made, such lesions alter the conductive characteristics of portions of the patient's heart, thereby controlling the symptoms of arrhythmias, such as supra-ventricular tachycardia, ventricular tachycardia, atrial flutter, atrial fibrillation, and other arrhythmias.

Such cardiac electrode catheters are typically placed within a desired portion of the patient's heart or arterial system by making a small incision in the patient's body at a location where a suitable artery or vein is relatively close to the patient's skin. The catheter is inserted through the incision into the artery and manipulated into position by threading it through a sequence of arteries, which may include branches, turns and other obstructions.

Once the cardiac electrode catheter has been maneuvered into the region of interest, one or more electrodes at the distal end of the catheter are placed against the anatomical feature or area sought to be diagnosed or treated. This can be a difficult procedure. The electrophysiologist manipulating the catheter typically can only do so by operating a system of controls at the proximal end of the catheter shaft. The catheter can be advanced and withdrawn longitudinally by pushing and pulling on the catheter shaft, and can be rotated about its axis by rotating a control at the proximal end. Both of these operations are rendered even more difficult by the likelihood that the catheter must be threaded through an extremely tortuous path to reach the target area. To facilitate maneuvering through tight and sinuous sequences of arterial or venous passageways, catheters have been developed with a predetermined portion of their distal ends having pre-shaped curves or dynamically alterably curves. However, the length of the distal end subject to curvature is fixed. As a result, a family of related catheters are developed with the primary difference between each family being the length of the curvable distal end. Variations in the length of the curvable distal ends provide variations in the curve radus. The range of radius is usually defined by the intended anatomical location and patient-to-patient variation. In order to change the curve radius during a procedure, a new member of the catheter family must be used. As a result, the electrophysiologist using the catheter may be required to make an alternative choice during the procedure if the originally selected fixed curve radius device is inappropriate to reach the desired location. This increases the length of the procedure and thereby the risk to the patient. Accordingly, there is a need for improving the navigation of the catheter to the treatment site by avoiding switching catheter devices in order to obtain a different curve radius.

Finally, once the tip of the catheter has reached the target area, the electrodes at the distal end of the catheter are placed in proximity to the anatomical feature, and diagnosis or treatment can begin. At this point, the electrophysiologist faces another difficultly of establishing and maintaining good contact with the treatment site tissue because only the most distal point of the electrode is likely to make contact with the tissue. Therefore, there is a need to improve the contact that a distal tip electrode makes with the treatment site.

Another use for electrode tip catheters is to produce linear-type lesions. Where the electrode is fixed to the end of a catheter, the manner of producing a linear-type lesion is to drag the catheter either proximally or distally from the original treatment site in order to produce a linear lesion. However, due to the unpredictable anatomy at the treatment site and along the passageway to which the remainder of the catheter is exposed, a linear lesion can be prevented because of unpredictable movement of the catheter distal end. In addition, to create a continuous lesion, the clinician must be careful not to move the catheter too far between successive ablations. If the clinician should accidentally move the catheter too far, then the lesion created will not be continuous, and the aberrant pathway may not be destroyed, requiring that the patient undergo yet another procedure, which is inefficient and undesirable. Accordingly, it is apparent that there continues to be a need for a device for performing ablations which ensures the creation of accurate linear lesions.

### SUMMARY OF EMBODIMENTS OF THE INVENTION

It is an object of an embodiment of this invention to improve the maneuverability of catheters through the tortuous arterial or venous passageways to a treatment site by providing a telescoping tip electrode which can protrude or extend from, or in an alternative, retract into a stabilized main catheter.

It is an object of an embodiment of this invention to provide that the mandrel on which the telescoping tip is attached and which extends from and retracts to the main catheter body is flexible. As a result, if the mandrel is extended during delivery of the telescoping tip electrode catheter to the treatment site, the flexibility of the mandrel can assist in maneuvering the passageways. In an alternative embodiment, the mandrel on which the telescoping tip is mounted need not be flexible, but rather can be inflexible.

It is a further object of this invention to improve tissue contact based on the telescoping tip electrode in combination with the telescoping tip portion on which the tip is mounted being made of flexible material and a portion of the catheter proximal of the telescoping tip portion being steerable. Therefore, when the telescoping tip is extended at any distance from the catheter main body and the steerable portion is manipulated to form a curve, the curve portion applies downward pressure to the extended electrode, thereby causing the extended electrode to flex downward against the cardiac tissue to order to improve contact of the electrode with the treatment site. In an alternative embodiment, the catheter main body proximal of the telescoping portion can be a preformed curve, which applies pressure to the telescoping tip when extended from the main catheter body and applied to the tissue.

It is another object of this invention to provide a linear-type lesion based on a predictable linear path of the tip electrode during extension from and retraction into the main catheter body.

### BRIEF DESCRIPTION OF THE DRAWINGS

Other objects and feature of the present invention will become apparent from the following detailed description of the preferred embodiment considered in conjunction with the accompanying drawings. It is understood, however, that the drawings are designed solely for the purposes of illustration and not as a definition of the limits of the invention.

FIGS. 1A to 1C are three perspective views of a portion of a telescoping tip electrode catheter with a steerable portion of the main catheter body just proximal of the telescoping tip portion according to an embodiment of the present invention;

FIG. 2 is a side view of the handle portion and the distal portion including the telescoping tip electrode and steerable portion of the catheter main body according to the FIGS. 1A to 1C embodiments;

FIG. 3 is an exploded perspective view of the telescoping tip electrode according to the FIGS. 1A to 1C embodiments;

FIG. 4 is a first partial cross sectional view of the telescoping tip electrode according to the FIGS. 1A to 1C embodiments;

FIG. 5 is a second partial cross sectional view of the telescoping tip electrode according to the FIGS. 1A to 1C embodiments;

FIG. 6 is a side view of the telescoping tip portion including the mandrel on which the telescoping tip electrode is mounted and the portion of the catheter main body just proximal of the telescoping tip portion according to the FIGS. 1A to 1C embodiments;

FIG. 7 is a partial cross section of the proximal portion of the main catheter body according to the FIGS. 1A to 1C embodiments;

FIG. 8 is a partial cross section of the telescoping tip electrode catheter showing the steering cables for the steerable catheter portion according to the FIGS. 1A to 1C embodiments;

FIG. 9 is a partial cross section of the telescoping tip electrode catheter showing the steering cables and the steerable catheter portion engaged in a curve according to the FIGS. 1A to 1C embodiments; and

FIG. 10 is a perspective view of the telescoping tip electrode catheter with the steerable portion engaged in a curve and the telescoping tip contacting a treatment site according to the FIGS. 1A to 1C embodiments.

### DETAILED DESCRIPTION OF THE PREFERRED EMBODIMENT

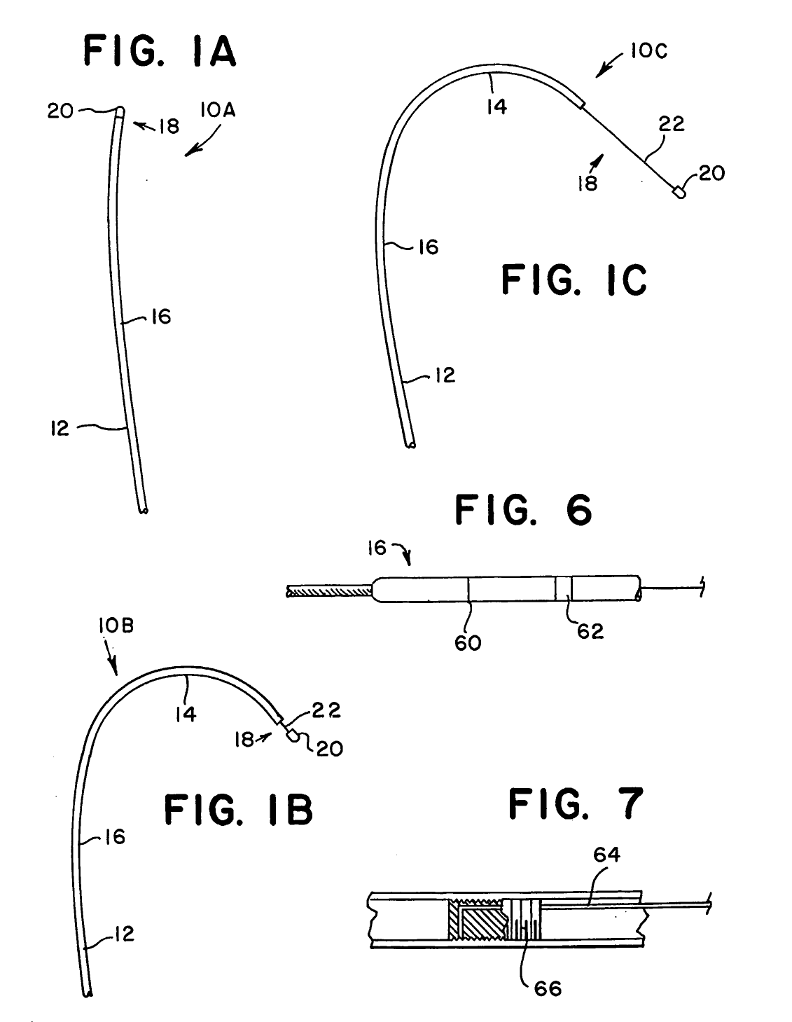

FIGS. 1A to 1C are three perspective views 10A, 10B and 10C, respectively, of a portion of a telescoping tip electrode catheter 11 with a steerable portion 14 of the main catheter body 16 just proximal of the telescoping tip portion 18 according to an embodiment of the present invention. More particularly, FIG. 1A shows the catheter 12 including a main body portion 16 and a telescoping tip portion 18. The telescoping tip portion of this FIG. 1A also shows the tip electrode 20. FIG. 1B shows the steerable portion 14 of the main catheter body 16 just proximal of the telescoping tip portion 18. The portion 18 includes a partially extended mandrel 22 which extends from and retracts to the main catheter body 16 and to which the telescoping tip electrode 20 attaches. FIG. 1C shows the steerable portion 14 engaged in a curve with a greater degree of curvature than the catheter portion 10B and the mandrel 22 extended to a greater length than the mandrel 22 in the catheter portion 10B. In alternative embodiments of the present invention, the tip electrode 22 can be retracted inside the catheter main body 16 rather than being external to the catheter main body 16 when the mandrel 22 is retracted to its full extent.

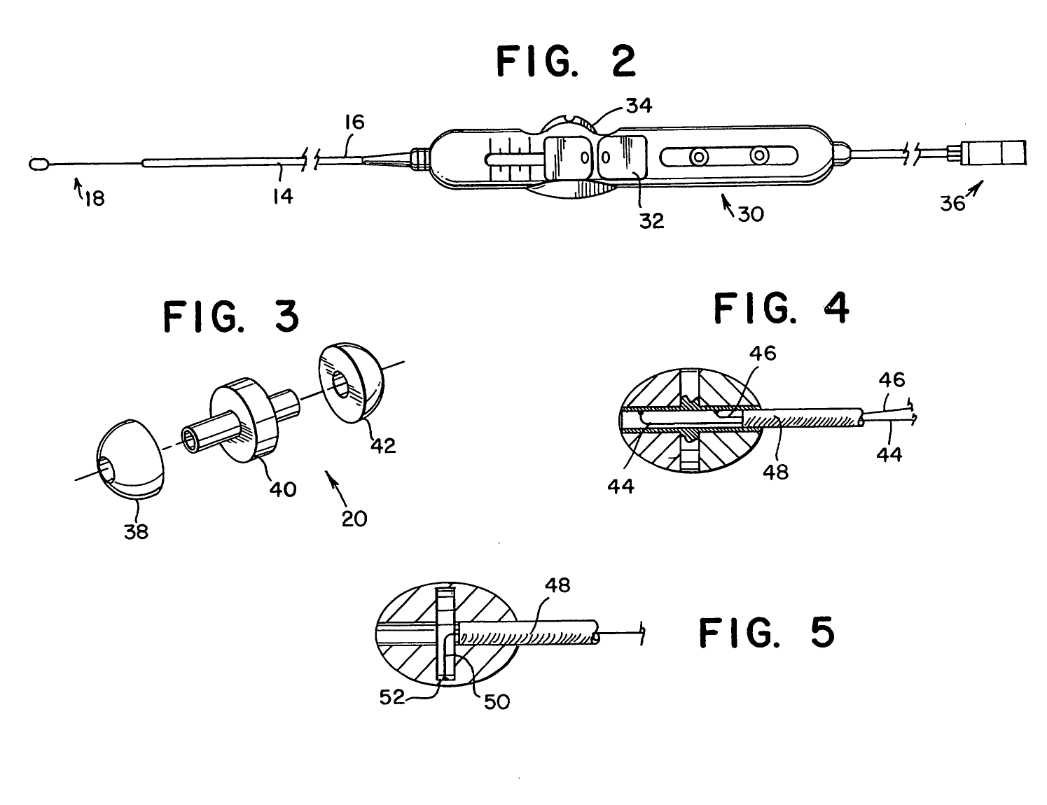

FIG. 2 is a side view of the handle portion 30 and the distal portion of the catheter main body 16 including the telescoping tip electrode 20 and the steerable portion 14 of the catheter main body 16 according to the FIGS. 1A to 1C embodiments. The handle portion 30 includes a slider mechanism 32 which operates the telescoping tip 18. The mechanism 32 moves in increments along the longitudinal axis of the catheter 10 and is connected in the interior (not shown) of the catheter 10 to the mandrel 22 for the telescoping tip 20. Movement of the slider mechanism 32 in either direction similarly causes the mandrel 22 to move in the same direction in order to extend or retract the tip electrode 20. For example, movement of the slider mechanism 32 proximally causes the mandrel 22 and the tip electrode 20 to retract and movement of the slider mechanism 32 distally causes the mandrel 22 and the tip electrode 20 to extend. The mechanism 32 can also be manipulated to cause partial movement of the mandrel 22 and tip electrode 20 so that partial extension at varying lengths of the tip electrode 20 can be achieved. A slider mechanism which can be used for an embodiment of the present invention is also disclosed in U.S. Pat. No. 6,178,354, to Charles Gibson arid issued on Jan. 23, 2001, which is incorporated herein in its entirety by reference. The handle portion 30 also includes a thumbwheel 34 which operates the steerable portion 14 of the catheter main body. The thumbwheel 34 and operation of the steerable portion 14 is described in U.S. Pat. No. 5,611,777, to Bowden et al. and issued on Mar. 18, 1997, which is incorporated herein in its entirety by reference. The handle portion 30 also connects to a generator device 36 which is proximal of the portion 30. The generator device portion 36 is used in a conventional manner to connect to a wire which carries power to the tip electrode 20. Such device 36 and operation is well known to those of ordinary skill in the art and therefore will not be further described herein.

FIG. 3 is an exploded perspective view of the telescoping tip electrode 20 according to the FIGS. 1A to 1C embodiments. In this embodiment, a bipolar electrode 20 is used, including three interlocking portions 38, 40 and 42. Portions 38 and 42 provide an elliptical shape to the electrode 20 and are the conducting portions. Portion 40 can be an electrical insulation. Exemplary materials for the construction of the electrode 2 are platinum, platinum/iridium or gold, etc. An exemplary size of the electrode 20 is 9 French with a length which can vary between about 4 to 8 mm. In alternative embodiments, the electrode 20 size can be smaller than the outer diameter of the main catheter body 16 so that the electrode 20 can retract inside the catheter 10. In further alternative embodiments, the electrode 20 can be a split electrode or any other type of shape (e.g., square, rectangular or circular) electrode 20 operable to treat tissue in a cardiac or arterial passageway.

FIG. 4 is a first partial cross sectional view of the telescoping tip electrode 20 according to the FIGS. 1A to 1C embodiments. The electrode 20 includes conductors 44 and 46 which provide power to the portions 38 and 42. Conductors 44 and 46 extend through the mandrel 22 to the generator device 36 (shown in FIG. 2). Also shown is a soldering bonding junction 48 between the electrode 20 and the mandrel 22.

FIG. 5 is a second partial cross sectional view of the telescoping tip electrode 22 according to the FIGS. 1A to 1C embodiments. Shown are a temperature sensor 50 and circumferential grove 52 around the electrode 20 for sensor placement. The soldering junction 48 is also shown between the electrode 20 and the mandrel 22. Referring also to FIGS. 1C and 2, an exemplary material for the mandrel 22 is nitinol, MP35N and SST. In alternative embodiments, where the mandrel 22 is not the electrical conductor, the material choices can be expanded to include non-conductive plastics that are durable but flexible, such as polyimide, PEEK or nylon, etc. In one embodiment, the length of the mandrel 22 and telescoping tip 20 portion 18 which extends or retracts from the main catheter body 16 can range in length from greater than 0 cm to about 6 cm or more in length. The diameter of the mandrel 22 can be 7 French for example. In alternative embodiments, the mandrel 22 diameter can be just smaller than the inner diameter of the main catheter body 16 shaft.

FIG. 6 is a side view of the telescoping tip portion 18 including the mandrel 22 on which the telescoping tip electrode 20 is mounted (not shown) and the portion of the catheter main body 16 just proximal of the telescoping tip portion 18 according to the FIGS. 1A to 1C embodiments. The portion of the main catheter body 16 includes a bonding area 60 in which the mechanisms to add in the steerability of the catheter 10 reside. Also shown in this embodiment is a ring electrode 62 for use in bipolar recordings, as is conventional. FIG. 7 is a partial cross section of the proximal portion of the main catheter body 16 according to the FIGS. 1A to 1C embodiments which shows the bonding area 60 in more detail. More particularly, the area 60 includes a steering anchor 64 and a threaded core assembly 66 for use in controlling the steerable portion 14 of the main catheter body 16.

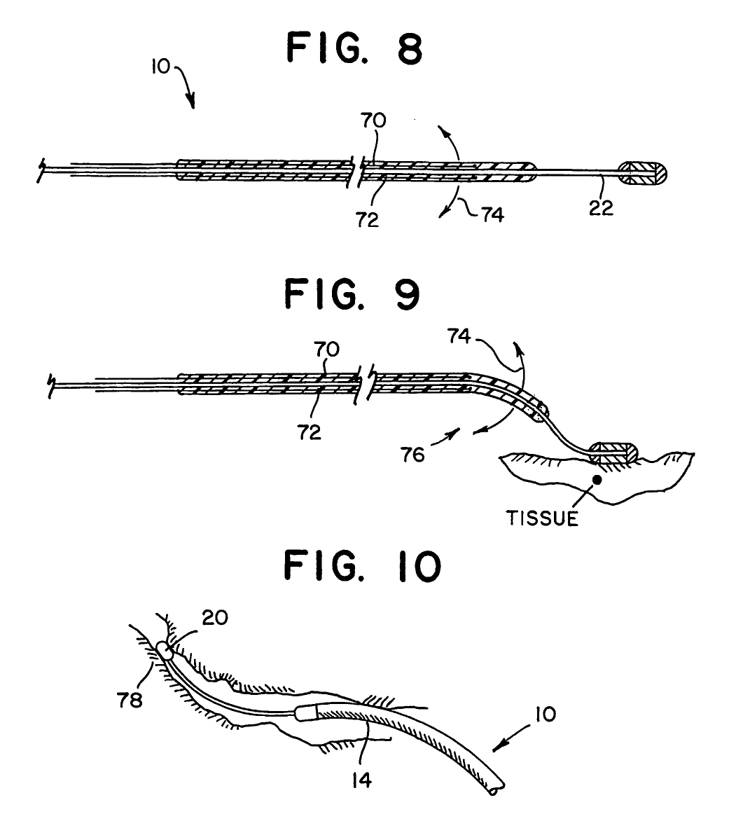

FIG. 8 is a partial cross section of the telescoping tip electrode catheter 10 showing the steering cables 70 and 72 for the steerable catheter portion 14. In this embodiment, the steerable portion 14 is located proximal of the distal end of the main catheter body 16. However, in alternative embodiments, the steerable portion 14 can extend to the distal end of the catheter main body 16. Curve directional arrows 74 show the potential direction of curvature for the steerable portion 14 in this embodiment. Also shown is mandrel 22 extending through the main catheter body 16 to connect to the slider mechanism 32, as described in U.S. Pat. No. 6,178,354, as cited above.

FIG. 9 is a partial cross section of the telescoping tip electrode catheter 10 showing the steering cables 70 and 72 and the steerable catheter portion 14 engaged in a curve 76 according to the FIGS. 1A to 1C embodiments.

FIG. 10 is a perspective view of the telescoping tip electrode catheter 10 with the steerable portion 14 engaged in a curve and the telescoping tip 20 being extended and contacting a treatment site 78 according to the FIGS. 1A to 1C embodiments. As a result of the curvature in the steerable portion 14, additional pressure is applied to the electrode 20 to improve the contact between the electrode 20 and the treatment site 78.

I claim:

1. A catheter for mapping and/or ablating intracardiac tissue comprising:
a catheter shaft for deployment into a desired intracardiac region for application of a linear lesion having at least one lumen therethrough and a distal end, the catheter shaft distal end being non-conductive; and
a telescoping mandrel extending through the lumen and including a distal end and a tip electrode at the mandrel distal end, the telescoping mandrel being moveable relative to the catheter shaft so as to extend from the distal end of the catheter shaft, thereby enabling the tip electrode to be positioned at a distance from the distal end of the catheter shaft.

2. The catheter of claim 1 wherein when the telescoping mandrel is extended from the catheter shaft distal end, the tip electrode is distal of its location when the telescoping mandrel is not extended from the catheter shaft distal end.

3. The catheter of claim 1 wherein the telescoping mandrel is one of flexible and inflexible.

4. The catheter of claim 1 wherein the location of the telescoping tip when the telescoping mandrel is not extended is one of housed within the catheter shaft and extended beyond the distal end of the catheter shaft.

5. The catheter of claim 1 further comprising:
the telescoping mandrel having a proximal end and the catheter shaft having a longitudinal axis;
a slider mechanism connected to the proximal end of the telescoping tip, the slider mechanism being movable along the longitudinal axis of the catheter shaft in order to extend the telescoping mandrel.

6. The catheter of claim 5 further comprising:
the slider mechanism being movable in increments along the longitudinal axis of the catheter shaft in order to incrementally control the extension of the telescoping mandrel.

7. A medical device comprising:
a catheter shaft having at least one lumen therethrough and a distal end; and
a telescoping mandrel extending through the lumen and including a distal end and a tip electrode at the mandrel distal end, the telescoping mandrel being moveable relative to the catheter shaft to extend from the catheter shaft distal end, thereby positioning the tip electrode at a distance from the catheter shaft distal end, wherein the tip electrode is constructed using one of platinum, platinum/iridium and gold.

8. A medical device comprising:
a catheter shaft having at least one lumen therethrough and a distal end; and
a telescoping mandrel extending through the lumen and including a distal end and a tip electrode at the mandrel distal end, the telescoping mandrel being moveable relative to the catheter shaft to extend from the catheter shaft distal end, thereby positioning the tip electrode at a distance from the catheter shaft distal end, wherein the size of the tip electrode is 9 French with a length of between 4 to 8 millimeters.

9. A medical device comprising:
a catheter shaft having at least one lumen therethrough and a distal end; and
a telescoping mandrel extending through the lumen and including a distal end and a tip electrode at the mandrel distal end, the telescoping mandrel being moveable relative to the catheter shaft to extend from the catheter shaft distal end, thereby positioning the tip electrode at a distance from the catheter shaft distal end, wherein the tip electrode is one of a bipolar electrode including three interlocking portions, the outer portions being conducting and the inner portion being insulating, a square electrode, a rectangular electrode and a circular electrode.

10. A medical device comprising:
a catheter shaft having at least one lumen therethrough and a distal end; and
a telescoping mandrel extending through the lumen and including a distal end and a tip electrode at the mandrel distal end, the telescoping mandrel being moveable relative to the catheter shaft to extend from the catheter shaft distal end, thereby positioning the tip electrode at a distance from the catheter shaft distal end, wherein the mandrel is constructed using one of nitinol, MP35N, SST, polyimide, PEEK and nylon.

11. The catheter of claim 1 further comprising the telescoping mandrel being further moveable relative to the catheter shaft to retract toward the catheter shaft distal end, thereby positioning the tip electrode one of adjacent to the catheter shaft distal end and inside the catheter shaft lumen.

12. The catheter of claim 2 wherein the telescoping tip of the telescoping mandrel in retracted position is one of housed within the catheter shaft and extended beyond the distal end of the catheter shaft.

13. The catheter of claim 2 further comprising:
the telescoping mandrel having a proximal end and the catheter shaft having a longitudinal axis;
a slider mechanism connected to the proximal end of the telescoping tip, the slider mechanism being movable along the longitudinal axis of the catheter shaft in order to extend and to retract the telescoping mandrel.

14. The catheter of claim 13 further comprising:
the slider mechanism movable in increments along the longitudinal axis of the catheter shaft in order to incrementally control the extension and the retraction of the telescoping mandrel.

15. The catheter of claim 2 further comprising means for actuating the telescoping mandrel to extend the tip electrode from the catheter shaft and to retract the tip electrode toward the catheter shaft distal end.

16. A medical device comprising:
a catheter shaft having at least one lumen therethrough and a distal end; and
a telescoping mandrel extending through the lumen and including a distal end and a tip electrode at the mandrel distal end, the telescoping mandrel being moveable relative to the catheter shaft to extend from the catheter shaft distal end, thereby positioning the tip electrode at a distance from the catheter shaft distal end, wherein the telescoping mandrel is one of flexible and inflexible; and
said means for actuating the telescoping mandrel is a thumb wheel.

17. A catheter of claim 1 further comprising:
the catheter shaft further having a proximal end, a longitudinal axis and a second lumen parallel to the shaft longitudinal axis and including a deflectable tip portion in the area of the catheter shaft distal end, the tip portion including a distal end and a lumen therethrough;
a pull wire attached off the longitudinal axis of the deflectable tip portion near its distal end, the pull wire extending proximally through the tip portion lumen, through one of the catheter shaft lumen and the second lumen; and
the pull wire being moveable to retract toward the catheter shaft proximal end thereby deflecting the tip portion.

18. The catheter of claim 2 further comprising:
the catheter shaft further having a proximal end, a longitudinal axis and a second lumen parallel to the shaft longitudinal axis and including a deflectable tip portion in the area of the catheter shaft distal end, the tip portion including a distal end and a lumen therethrough;
a pull wire attached off the longitudinal axis of the deflectable tip portion near its distal end, the pull wire extending proximally through the tip portion lumen, through one of the catheter shaft lumen and the second lumen; and
the pull wire being moveable to retract toward the catheter shaft proximal end thereby deflecting the tip portion.

19. A medical device comprising:
a catheter shaft having at least one lumen therethrough and a distal end; and
a telescoping mandrel extending through the lumen and including a distal end and a tip electrode at the mandrel distal end, the telescoping mandrel being moveable relative to the catheter shaft to extend from the catheter shaft distal end, thereby positioning the tip electrode at a distance from the catheter shaft distal end;
a control handle having a longitudinal axis therethrough and a proximal end and a distal end;
a slide block axially movably supported in the control handle, the slide block having a distal part and a separate proximal part not fixedly connected to the slide block distal part, the distal part having an external helical thread;
the catheter shaft having a proximal end attached to the control handle distal end and having at least one passageway therethrough from the shaft proximal end to a shaft distal end, the shaft further including a deflectable tip portion located near the catheter shaft distal end, the tip portion also having a distal end and a longitudinal axis therethrough; and
a pullwire attached off-axis to the deflectable tip portion near its distal end, the pullwire extending proximally through the tip portion, through one of the catheter shaft lumen and a second lumen of the catheter shaft parallel to the catheter shaft longitudinal axis, into the control handle through its distal end, through an opening in the distal part of the slide block, and being secured to the proximal part of the slide block; and
an axially rotatably mounted thumb wheel surrounding the distal part of the slide block, the thumb wheel having an internal helical thread in engagement with the external helical thread on the distal part of the slide block, whereby deflection of the tip portion is caused by proximal displacement of the distal part of the slide block within the control handle, which is, in turn, caused solely by axial rotation of the thumb wheel.
